(19) United States
(12) Patent Application Publication
(10) Pub. No.: US 2005/0106694 A1
(43) Pub. Date: May 19, 2005

(54) Lactic acid production

(75) Inventors: Edward Green, Surrey (GB); Muhammad Javed, Essex (GB); Renia Gemmell, Oxford (GB)

Correspondence Address: BANNER & WITCOFF, 1001 G STREET N W, SUITE 1100, WASHINGTON, DC, 20001, US

(73) Assignee: Elsworth Biotechnology Ltd., Surrey (GB)

(21) Appl. No.: 10/484,035

(22) PCT Filed: Jul. 18, 2002
(86) PCT No.: PCT/GB02/03272

(30) Foreign Application Priority Data
Jul. 18, 2001 (GB) 0117551.2

(51) Int. Cl. C12P 7/42; C12N 1/21

(52) U.S. Cl. 435/146; 435/252.3

(57) ABSTRACT

The present invention relates to a bacterium capable of converting sugars into lactic acid or a salt thereof. The invention also relates to a method for producing lactic acid or a salt thereof comprising culturing the bacterium of the present invention. In particular, the present invention provides a thermophilic bacterium capable of converting at least 70% (w/w) of a monosaccharide sugar and a disaccharide sugar into lactic acid or a salt thereof.

The present invention relates to a bacterium capable of converting sugars into lactic acid or a salt thereof. The invention also relates to a method for producing lactic acid or a salt thereof comprising culturing the bacterium of the present invention.

Lactic acid is a versatile chemical, used as an acidulant, a flavouring and preservative in food, in pharmaceuticals, and in leather and textile industries. It is also used in the production of base chemicals and for polymerisation of biodegradable plastics.

Lactic acid exists as two optical isomers, D and L. Both isomeric forms of lactic acid can be polymerised and polymers with different properties produced. In particular, L-lactic acid forms the base of polyacrylate, polylactate and polylactide (polylactic acid) which are being used increasingly in the polymer industry.

Over 40,000 tons of lactic acid are produced worldwide every year and about two thirds are made by lactic acid bacterial fermentation. The rest is produced synthetically by the hydrolysis of lactonitrile.

Fermentative production has the advantage that depending on the strain of bacteria used, only one of the isomers of lactic acid is produced. With synthetic production a racemic mixture of lactic acid is produced. Fermentative lactic acid production comprises the pre-treatment of a suitable substrate (including hydrolysis to produce sugars), fermentation of the sugars to lactic acid, separation of bacteria and solid particles from the derived broth and purification of lactic acid. One of the current problems with the production of lactic acid is that substrate costs are a major element in the conventional fermentation process costs. This is because the sugars used are mostly derived from starch, sugar beet or sugar cane juice, that have high values as food.

Lactic acid production using strains of lactobacilli is described by Siebold et al (Process Biochemistry, 30, 81-95, 1995), wherein the cultivation media comprise glucose as one of the main sugar components. The lactobacilli used cannot use the full range of hexose and pentose sugars derived from cheaper feed stocks.

Cheaper feed stocks are usually agro-industrial waste streams such as from wet-milling of paper pulping, that are rich in pentose sugars and are of low or even negative commercial value. In addition there are enormous volumes of solid food processing wastes such as bran and shives from dry-milling, sugar cane bagasse, or oilseed processing residues etc., that are rich in hemicelluloses and that can be readily converted to a mixture of sugars by dilute acid or alkali hydrolysis. Such cheap crude feed stocks have not been widely exploited because the prior art industrial microorganisms cannot use them efficiently.

Danner el al (Applied Biochemistry and Biotechnology, 70-72, 895-903, 1998) describes the use of two different Bacillus stearothermophilus strains for the production of L-lactic acid. Both strains require complex media constituents including yeast extract and peptone. Danner et al (Biomass for Energy and Industry, 446-449, 1998) also discloses Bacillus stearothermophilus strains requiring complex growth media.

Datta et al (FEMS Microbiology Reviews, 16, 221-231, 1995) discusses the various technological and economic potential of the production of lactic acid and in particular discusses the production of lactic acid by Siebold et al (supra).

Rowe et al., (J. Bact., 124, 279-284, 1975) discloses Bacillus stearothermophilus strains that are capable of growth on a number of different carbon sources in a defined medium. There is no indication that the bacteria can be used to efficiently produce lactic acid. Furthermore, the strains have the drawback that they cannot grow on a number of carbon sources, including lactate and acetate.

All of the prior art methods for the production of lactic acid by fermentation either require relatively pure substrates and/or complex media for growth of the bacterial strain. There is a need for the production of lactic acid from substrates containing different sugars where the lactic acid is produced to a high level and is easy to purify. In particular, there is a need for broadening the substrate range of any bacterium used to produce lactic acid (especially so that it can utilise monosaccharide sugars, including pentose sugars, and disaccharide sugars), increasing the lactic acid tolerance of the bacterium and avoiding the addition of complex supplements to culture media.

The present invention overcomes at least some of the problems associated with the prior art strains used in the production of lactic acid.

The present invention provides a thermophilic bacterium capable of converting a monosaccharide sugar and a disaccharide sugar into lactic acid or a salt thereof, when grown in a defined medium, wherein at least 60% (w/w) of the monosaccharide sugar and the disaccharide sugar are converted into lactic acid or a salt thereof.

Accordingly, it is possible to use relatively crude substrates such as wood pulping wastes, wheat straw, wood chips, forestry wastes including prunings and other waste materials, sugar beat pulp, milling residues and brewer's spent grains, with the bacterium of the present invention for producing lactic acid or a salt thereof.

As the bacterium is capable of converting both monosaccharide sugars and disaccharide sugars into lactic acid it is capable of utilising substrates comprising one or both of these sugars efficiently in order to produce lactic acid or a salt thereof.

Suitable monosaccharide sugars include both pentose and hexose sugars. Preferably the monosaccharide sugar is selected from arabinose, fructose, glucose and xylose. It is particularly preferred the monosaccharide sugar is selected from glucose and xylose.

The disaccharide sugar is preferably selected from sucrose, lactose and cellobiose. It is further preferred that the disaccharide sugar is sucrose.

It is further preferred that the bacterium of the present invention is capable of utilising simultaneously two different sugars. It is particularly preferred that the bacterium is capable of utilising simultaneously xylose and glucose.

Salts of lactic acid include inorganic salts such as metals, organic salts and esters, for example, sodium lactate, magnesium lactate, calcium lactate, ammonium lactate and ethyl lactate.

The term “a defined medium” refers to a culture medium which does not contain any undefined components such as yeast extract, peptone, tryptone, other meat extracts and complex nitrogen sources. These components complicate purification and some are relatively expensive (e.g. yeast extract).

It is preferable to first develop a process medium using a defined chemical composition and then, if necessary, substitute any expensive nutrient supplements with cheap complex sources (if available) that do not interfere with purification.

It is preferred that the bacterium of the present invention is capable of growth in a medium comprising lactate and/or acetate as the sole carbon source.

The advantage of the bacterium being able to grow in a medium containing lactate and/or acetate as the sole carbon source is that the waste cell culture can be recycled (after cell removal) to grow fresh cell biomass. It may be necessary to alter the culture conditions to ensure that the bacteria of the present invention can utilise lactate and/or acetate as the carbon source, e.g. by vigorously sparging the medium with air so that aerobic growth of the bacteria occurs and by changing the pH of the culture.

The thermophilic bacterium may be any species of bacterium capable of converting a monosaccharide sugar and a disaccharide sugar into lactic acid or a salt thereof when grown on a defined medium. Preferably the thermophilic bacterium is a Bacillus sp. bacterium. Suitable Bacillus spp. include B. stearothermophilus; B. caldovelox; B. caldotenax; B. thermoglucosidasius; B. coagulans; B. licheniformis; B. thermodenitrificans; B. caldolyticus; B. smithii; and B. fumarioli. Preferably, the thermophilic bacterium of the present invention is Strain LN (NCIMB Accession number 41038; strain J44 (NCIMB Accession number 41111); strain J30 (NCIMB Accession number 41113); and strain SCM6 (NCIMB Accession number 41112).

Preferably the bacterium of the present invention is capable of converting a monosaccharide and a disaccharide sugar to lactic acid or a salt thereof at a pH of 5 to 9, more preferably at a pH of 6 to 8.

It is further preferred that the bacterium of the present invention is capable of growth in a defined medium at a pH of less than 7.0.

It is further preferred that the bacterium of the present invention is capable of converting at least 70% w/w of a monosaccharide and a disaccharide sugar into lactic acid. It is further preferred that the bacterium is capable of converting at least 80% w/w, more preferably 95% w/w of a monosaccharide and a disaccharide sugar into lactic acid or a salt thereof.

Preferably the bacterium of the present invention has an exponential growth rate (μ) greater than 1.0 (h⁻¹) in a defined medium. The exponential growth rate (μ) is calculated using the following formula.

$$μ = \frac{ln {X}_{T} - ln {X}_{O}}{( T - {T}_{O} )}$$

Bacterial concentrations X_{O} and X_{T} at times T_{O} and T.

Preferably at least 99% of the lactic acid produced by the bacterium of the present invention is the L optical isomer.

It is further preferred that the bacterium of the present invention is sporulation deficient.

Preferably the bacterium of the present invention is a facultative anaerobe.

The bacterium of the present invention can be obtained by screening a population of Bacillus strains to identify those strains having the required characteristics, namely, thermophilic, capable of converting a monosaccharide sugar and a disaccharide sugar into lactic acid or a salt thereof when grown on a defined medium. Suitable screening methods comprise determining cell growth and lactate production of bacteria on different carbon sources at high temperature (see Biomass for Energy and Industry, Danner et al., 446-449, 1998).

Preferred bacteria of the present invention have been deposited. Other bacteria of the present invention can therefore be obtained by mutating the deposited bacteria and selecting derived mutants having enhanced characteristics. Desirable enhanced characteristics include an increased range of sugars that can be utilised, increase growth rate, ability to produce lactic acid at a lower pH etc. Suitable methods for mutating bacteria and selecting desired mutants are described in Functional Analysis of Bacterial Genes: A Practical Manual, edited by W. Schumann, S. D. Ehrlich & N. Ogasawara, 2001.

The present invention also provides a method of producing lactic acid or a salt thereof comprising culturing the bacterium of the present invention in a culture medium under suitable conditions. Methods for culturing bacteria to produce lactic acid are well known to those skilled in the art. In particular, the method might comprise a continuous fermentation process, a batch fermentation process or a fed batch fermentation process. Preferably the method of the present invention comprises culturing the bacterium in a continuous fermentation process. Continuous fermentation processes are well known to those skilled in the art and are described in Principles of Microbe and Cell Cultivation, J. S. Pirt, Blackwell Scientific Publications, 1985. The advantages of continuous fermentation are reduced downtime and increased productivity.

Preferably the method of the present invention comprises sparging the culture medium with air so that the culture is microaerobic.

It is further preferred that the culture medium used in the method of the present invention is a defined culture medium.

It is further preferred that the culture medium used in the method of the present invention comprises lactate and/or acetate as the sole carbon source.

Preferably the method of the present invention is operated at a temperature of between 40 and 70° C., more preferably 50 and 65° C., and most preferably between 52 and 60° C.

Preferably the method of the present invention has a minimum productivity of lactic acid or salt thereof of 4.2 grams/litre of culture/hour.

The present invention is now described by way of example only.

### EXAMPLE

### Methods

Culture Conditions

Microaerophilic assays were performed in triplicate in 15 ml Falcon tubes for each isolate, with 1% sugar. Medium controls, with no added sugar, were included. Inocula were prepared by suspending cells from overnight plate cultures in the J-LD Minimal Medium; 500 μl cell suspension was added to each tube. Additional controls included J-LD Minimal Medium with sugar, but no inoculum. All tubes were incubated in shaker incubators for 2.5 h to bring the culture to the exponential phase of growth, before static incubation: J30 and LN were incubated at 60° C. whereas J44 and SCM6 were incubated at 52° C.

Aerobic assays were performed in duplicate 50 ml shake flasks with 0.5% acetate and 0.5% lactate. Medium controls, with 0% and 0.5% glucose were included. The flasks were inoculated with a 5% inoculum (2.5 ml) and incubated for 24 h. J30 and LN were incubated at 60° C. whereas J44 and SCM6 were incubated at 52° C. Cell growth was calculated by comparing the optical density (OD₆₀₀) of the cultures with the controls.

Composition of J-LD Minimal Medium (Per Litre)

Salts

NH₄Cl, 1 g; NaH₂PO₄, 0.5 g; MgSO₄.7H₂O, 0.2 g; KCl, 0.2 g; MnCl₂.4H₂O, 3 mg; CaCl₂.2H₂O, 5 mg.

Trace elements (0.25 ml)

ZnSO₄.7H₂O, 0.08 mg; Boric acid, 0.02 mg; CoCl₂.6 H₂O, 0.1 mg; Cu SO_{4.}5 H₂O, 0.4 mg; Fe Cl₃.6 H₂O, 1.075 mg; Ni Cl₂.6 H₂O 0.02 mg; with EDTA, 0.5 mg.

Amino acids

Aspartic acid, 0.3 g; glutamic acid, 0.6 g; isoleucine, 0.3 g; methionine, 0.3 g; serine, 0.3 g.

Vitamins

D-biotin, 2 mg; nicotinic acid, 3 mg; pyridoxine HCl, 0.9 mg; riboflavin, 0.9 mg; thiamin HCl, 2 mg.

Buffer Cascade

PIPES (pH 7.5), 40 mM; Bis-TRIS (pH 7.5), 40 mM; HEPES (pH 7.5), 40 mM.

Carbohydrates

Xylose, arabinose, glucose, fructose, sucrose, lactose, cellobiose and starch (10 g)

Oxidase

A portion of a colony was picked up with a plastic loop and smeared on a filter paper moistened with a 1% (w/v) solution of N,N,N′,N′-tetramethyl-p-phenylenediamine. Purple colouration within 10 seconds was taken to indicate the presence of oxidase enzymes.

Catalase

A loopful of colony was mixed in a drop of hydrogen peroxide (>30% w/v). Production of oxygen (effervescence) indicated the presence of catalase.

pH

Cultures for pH assay were set up in 15 ml Falcon tubes at set pH values. Tubes were inoculated with LN, J30 or J44 from flask cultures and incubated for 22 hours at 60° C. The SCM6 inoculum was prepared as a cell suspension and tubes were incubated at 52° C. for 24 hours.

Microscopy and Gram stain

Cell pellets were washed and re-suspended in quarter-strength Ringers solution. Gram stains were performed using the conventional procedure, applying the bioMérieux Gram Stain reagents for two minutes each (crystal violet solution, 2%; iodine solution, 1.3% I₂ and 2% KI; safranin solution, 0.25%) and acetone for 2-3 seconds.

PCR

The 16S rRNA genes of J30, J44 and SCM6 were amplified by colony PCR using primers 16S-A (5′ to 3′: CCG AAT TCG TCG ACA CAG TTT GAT CAT GGC TCA G) and 16S-B (5′ to 3′: CCC GGG ATC CAA GCT TAG AAA GGA GGT GAT CCA), with the following thermal cycling conditions: 94° C., for 5 minutes, then 30 cycles of 94° C. for 1 minute, 48° C. for 1 minute and 72° C. for 2 minutes, with a final elongation time of 10 minutes at 72° C. The PCR products were purified by agarose gel extraction (Qiagen). PCR products were sequenced by PNACL (University of Leicester).

Api 50 CHB test strips

The BioMérieux api 50 CH (carbohydrates) test strips with CHB (Bacillus) medium were used. These were incubated at 52° C. for J44 and SCM6 and at 60° C. for LN and J30, and the results noted at 4 hours and 24 hours.

Lactate assay

Lactate was measured using the SIGMA Lactate Assay and the purity of the isomer was measured using the Roche D-Lactic acid/L-Lactic acid analysis kit in accordance with the manufacturers' recommendations.

### Strain Characterisation

Sugar Utilisation

In the sugar assay, strain LN utilised and produced lactate from xylose, arabinose, glucose, fructose, sucrose and cellobiose. This strain also utilised xylose and glucose simultaneously. Results from the Api 50 CHB test showed that LN also utilised ribose, D-mannose, maltose, saccharose, trehalose, D-raffinose, D-turanose, α-methyl-D-glucoside, n-acetyl glucosamine, arbutine and salicine.

In the sugar assay, strain J30 utilised and produced lactate from xylose, glucose, fructose and sucrose. Results from the Api 50 CHB test showed that J30 also utilised glycerol, ribose, galactose, D-mannose, mannitol, α-methyl-D-glucoside, maltose, saccharose, trehalose and D-turanose.

In the sugar assay, strain J44 utilised and produced lactate from xylose, arabinose, glucose, fructose, sucrose, cellobiose and lactose. Results from the Api 50 CHB test showed that J44 also utilised glycerol, ribose, galactose, D-mannose, rhamnose, α-methyl-D-mannoside, α-methyl-D-glucoside, N-acetyl glucosamine, amygdaline, arbutine, esculine, salicine, maltose, melibiose, saccharose, trehalose, D-raffinose, amidon, β-gentiobiose, D-turanose and gluconate.

In the sugar assay, strain SCM6 utilised and produced lactate from xylose, arabinose, glucose, fructose, sucrose, lactose and starch. Results from the Api 50 CHB test strip showed that SCM6 also utilised glycerol, ribose, galactose, D-mannose, L-sorbose, inositol, mannitol, sorbitol, α-methyl-D-glucoside, amygdaline, arbutine, esculine, salicine, maltose, saccharose, trehalose, glycogene, D-turanose and cellobiose.

Strains LN, J30, J44 and SCM6 all grew aerobically on lactate (0.5% (w/v)) and acetate (0.5% (w/v)). The results are shown in Table 2.

TABLE 1
Strain Characteristics

| | LN | J30 | J44 | SCM6 |
|---|---|---|---|---|
| Identification | B. thermoglucosidasius | B. smithii | B. coagulans | B. licheniformis |
| Colony form | Low convex, | To 7 mm; | To 2 mm; | Pale pink/white |
| | smooth, sometimes | spreading; | smooth, | spreading, |
| | crenated; to 2 mm | very mucoidal | entire, slightly | licheniform dry |
| | | | umbonate; | and rough |
| | | | waxy | surface slime |
| Cell shape | Rod, 3-5 μm; motile | Rod, 2-5 μm; | Rod, 3-5 μm; | Rods, varied |
| | | some chains; | some long | lengths 2-9 μm; |
| | | | chains; motile | some chains |
| | | | | motile |
| Sporulation | No | Yes | — | — |
| Gram reaction | Gram positive | Gram positive | Gram positive | Gram positive |
| Oxidase | Weak positive | Weak positive | Negative | Negative |
| Catalase | Positive | Weak positive | Strong positive | Positive |
| Temperature range | 50° C.-70° C. | 37° C.-70° C. | 37° C.-65° C. | 25° C.-52° C. |
| Optimum | 65° C. | 60° C. | 52° C. | — |
| temperature | | | | |
| PH range | 6.2-9 | 6-9 | 5-9 | 5-9 |
| Optimum pH | pH 7.0 | pH 7.0 | pH 8.0 | — |
| Growth rate (h⁻¹) | >2.0 | c.1.8 | c.1.3 | — |
| Lactate yield | 0.7 | 0.7 | 0.9 | — |
| (g/g sugar) | | | | |
| L-Lactate Purity | 99.2% | 99.6% | 99.7% | — |

TABLE 2

| Aerobic growth on | LN | J30 | J44* | SCM6 |
|---|---|---|---|---|
| Lactate (0.5% w/v) | ++++ | ++ | + | +++ |
| Acetate (0.5% w/v) | ++ | + | + | ++ |

++++ heavy growth
+++ moderate growth
++ light growth
+ poor growth
*J44 is a slow-growing microorganism, hence poor growth on both substrates.

1. A thermophilic bacterium capable of converting a monosaccharide sugar and a disaccharide sugar into lactic acid or a salt thereof, when grown in a defined medium, wherein at least 60% (w/w) of the monosaccharide sugar and the disaccharide sugar are converted into lactic acid or a salt thereof.

2. The bacterium of claim 1, wherein the monosaccharide sugar is a pentose and/or a hexose sugar.

3. The bacterium of claim 1 or claim 2, wherein the monosaccharide sugar is selected from the group consisting of arabinose, fructose, glucose, and xylose.

4. The bacterium of claim 3, wherein the monosaccharide sugar is selected from the group consisting of glucose and xylose.

5. The bacterium of anyone of the previous claims claim 1, wherein the disaccharide sugar is selected from the group consisting of sucrose, lactose, and cellobiose.

6. The bacterium of claim 5, wherein the disaccharide sugar is sucrose.

7. The bacterium of claim 1, which is capable of utilising simultaneously two different sugars.

8. The bacterium of claim 7, wherein the two different sugars are xylose and glucose.

9. The bacterium of claim 1, which is capable of growth in a medium comprising lactate and/or acetate as the sole carbon source.

10. The bacterium of claim 1, wherein the bacterium is a Bacillus sp. bacterium.

11. The bacterium of claim 10, wherein the Bacillus is selected from B. stearothermophilus; B. caldovelox; B. caldotenax; B. thermoglucosidasius; B. coagulans; B. licheniformis; B.thermodenitrificans; B. caldolyticus; B. smithii; and B. fumarioli.

12. The bacterium of anyone claim 1, which is capable of converting the monosaccharide and the disaccharide sugar to lactic acid or a salt thereof at a pH of 5 to 9.

13. The bacterium of claim 12, which is capable of converting the monosaccharide and the disaccharide sugar to lactic acid or a salt thereof at a pH of 6 to 8.

14. The bacterium of claim 1, which is capable of growth at a pH of less than 7.0.

15. The bacterium of claim 1, wherein at least 70% w/w of monosaccharide and disaccharide sugars are converted into lactic acid or salt thereof.

16. The bacterium of claim 1, wherein at least 80% w/w of the monosaccharide and disaccharide sugars are converted into lactic acid or salt thereof.

17. The bacterium of claim 1, wherein at least 95% w/w of the monosaccharide and disaccharide sugars are converted into lactic acid or salt thereof.

18. The bacterium of claim 1, which has an exponential growth rate (μ) greater than 1 h⁻¹ in a defined medium.

19. The bacterium of claim 1, wherein at least 99% of the lactic acid produced is the L-optical isomer.

20. The bacterium of claim 1, which is sporulation deficient.

21. The bacterium of claim 1, wherein the bacterium is a facultative anaerobe.

22. A bacterial strain selected from the group consisting of strain LN (NCIMB Accession number 41038; strain J44 (NCIMB Accession number 41111); strain J30 (NCIMB Accession number 41113); and strain SCM6 (NCIMB Accession number 41112).

23. A method of producing lactic acid or a salt thereof comprising culturing the bacterium of claim 1 in a culture medium under suitable conditions.

24. The method of claim 23, wherein the culturing is performed in a continuous fermentation process.

25. The method of claim 23, wherein the culture medium is sparged with air and the culture is microaerobic.

26. The method of claim 23, wherein the culturing is at a temperature of between 40 and 70° C.

27. The method of claim 23, wherein the culturing is at a temperature of between 50 to 65° C.

28. The method of claim 23, wherein the culturing is at a temperature of between 52 and to 60° C.

29. The method of anyone of claim 23, wherein the bacterium produces at least 4.2 g/litre of culture/hour of lactic acid or a salt thereof.

30. The method of anyone of claim 23, wherein the culture medium comprises lactate and/or acetate as the sole carbon source.
